(19) United States
(12) Patent Application Publication
(10) Pub. No.: US 2013/0085396 A1
(43) Pub. Date: Apr. 4, 2013

(54) ULTRASONIC PROBE AND ULTRASONIC DISPLAY DEVICE

(71) Applicant: GE MEDICAL SYSTEMS GLOBAL TECHNOLOGY COMPANY, LLC, Waukesha, WI (US)

(72) Inventors: Hiroshi ISONO, Tokyo (JP); Masaaki OHTSUKA, Tokyo (JP)

(73) Assignee: GE MEDICAL SYSTEMS GLOBAL TECHNOLOGY COMPANY, LLC, Waukesha, WI (US)

(21) Appl. No.: 13/629,313

(22) Filed: Sep. 27, 2012

(30) Foreign Application Priority Data
Sep. 29, 2011 (JP) 2011-215098

(51) Int. Cl. A61B 8/00; B23P 17/04; B23P 11/00

(52) U.S. Cl. 600/472; 29/428; 29/458

(57) ABSTRACT

An ultrasonic probe is provided. The ultrasonic probe includes a reflection layer between an ultrasonic transducer and a backing layer, the reflection layer configured to reflect an ultrasonic wave transmitted from the ultrasonic transducer, wherein the backing layer includes a backing material, and wherein a thermally-conductive layer of material having a thermal conductivity higher than that of the backing material is formed over a surface of the backing layer.

### CROSS REFERENCE TO RELATED APPLICATIONS

This application claims the benefit of Japanese Patent Application No. 2011-215098 filed Sep. 29, 2011, which is hereby incorporated by reference in its entirety.

### BACKGROUND OF THE INVENTION

The present invention relates to an ultrasonic probe and an ultrasonic display device which incorporates a measure against the heat generated in an ultrasonic transducer.

An ultrasonic display device displays an ultrasonic image based on echo signals obtained by ultrasonically scanning a subject. Such an ultrasonic display device performs ultrasonic scanning using an ultrasonic probe connected thereto via a probe cable.

The ultrasonic probe has an ultrasonic transducer, an acoustic matching layer and a backing material. More specifically, the ultrasonic transducer is provided with the acoustic matching layer on the object side and the backing material on the side opposite to the object side (see, for example, JP-A No. 2009-61112). On the subject side of the acoustic matching layer, an acoustic lens to be in contact with the subject is provided. The ultrasonic transducer includes a piezoelectric element of, for example, zirconate titanate (PZT). A voltage is applied to the ultrasonic transducer to transmit ultrasonic waves.

When ultrasonic waves are transmitted and received, heat is generated in the ultrasonic transducer. Since the backing material has a thermal conductivity lower than that of the acoustic matching layer, the heat generated in the ultrasonic transducer is conducted not to the backing material side but to the acoustic matching layer side, i.e. to the subject side. Hence, when the ultrasonic probe is kept in use, the surface temperature of the acoustic lens rises. When ultrasonic waves are transmitted/received, therefore, the ultrasonic output of the ultrasonic transducer is limited so as to prevent excessive rising of the surface temperature of the acoustic lens. Hence, ultrasonic probes in which the heat generated in an ultrasonic transducer can be released toward the side opposite to the subject side are desired.

### BRIEF DESCRIPTION OF THE INVENTION

An ultrasonic probe is provided. The ultrasonic probe having includes a reflection layer between an ultrasonic transducer and a backing layer, the reflection layer being for reflecting ultrasonic waves transmitted from the ultrasonic transducer. In the ultrasonic probe, the backing layer includes a backing material over a surface of which a thermally-conductive layer of material having a thermal conductivity higher than that of the backing material is formed.

According to the ultrasonic probe described above, the thermally-conductive layer is formed over the surface of the backing material, so that the heat generated in the ultrasonic transducer can be released to the side opposite to the subject side. Since the ultrasonic waves transmitted from the ultrasonic transducer are reflected not by the thermally-conductive layer but by the reflection layer, the transmission of ultrasonic waves to the subject side is prevented from being adversely affected acoustically.

### BRIEF DESCRIPTION OF THE DRAWINGS

FIG. 1 is a block diagram showing an exemplary embodiment of the ultrasonic diagnostic apparatus.

FIG. 2 is an external perspective view of an ultrasonic probe.

FIG. 3 is an external perspective view of only the functional element section of the ultrasonic probe shown in FIG. 2.

FIG. 4 is a sectional view of the functional element section of the ultrasonic probe shown in FIG. 2.

FIG. 5 is a sectional view of another example of a backing layer.

FIG. 6 is a diagram for explaining ultrasonic wave transmission.

FIG. 7 is a diagram for explaining the ratio between the widths in the x-axis direction of a thermally-conductive layer and backing material.

### DETAILED DESCRIPTION OF THE INVENTION

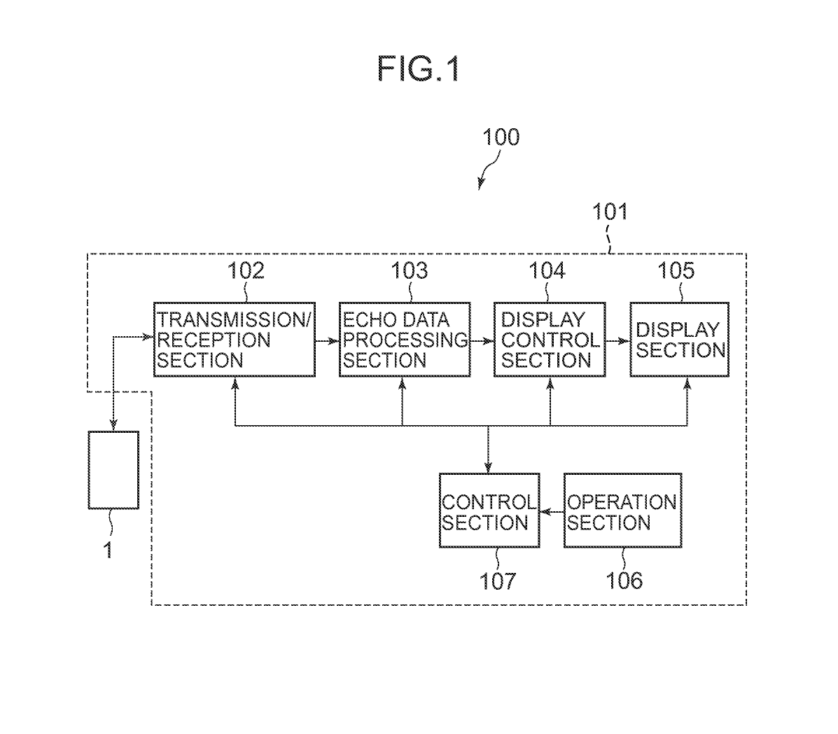

An exemplary embodiment will be described below. An ultrasonic diagnostic apparatus 100 shown in FIG. 1 is an example of an ultrasonic display device and includes an ultrasonic probe 1 and an apparatus main body 101 to which the ultrasonic probe 1 is connected.

The apparatus main body 101 is provided with a transmission/reception section 102, an echo data processing section 103, a display control section 104, a display section 105, an operation section 106, and a control section 107.

The transmission/reception section 102 supplies an electrical signal used to transmit an ultrasonic wave from the ultrasonic probe 1 under predetermined scanning conditions to the ultrasonic probe 1 based on a control signal received from the control section 107. The transmission/reception section 102 also processes an echo signal received by the ultrasonic probe 1, for example, for A/D conversion or phase rectifying addition.

The echo data processing section 103 processes echo data outputted from the transmission/reception section 102 to generate an ultrasonic image. For example, the echo data processing section 103 generates B-mode data by performing B-mode processing such as logarithmic compression and envelope detection.

The display control section 104 generates ultrasonic image data by scan-converting data inputted from the echo data processing section 103 using a scan converter and displays an ultrasonic image based on the ultrasonic image data on the display section 105. The display control section 104 generates, for example, B-mode image data based on B-mode data and displays a B-mode image on the display section 105.

The display section 105 includes, for example, a liquid crystal display (LCD) or a cathode ray tube (CRT). The operation section 106 includes, for example, switches, a keyboard and a pointing device (not shown) for use by an operator to input commands and information.

The control section 107 includes a central processing unit (CPU), not shown. The control section 107 reads a control program stored in a storage section, not shown, and causes functions of various sections of the ultrasonic diagnostic apparatus 100 to be performed.

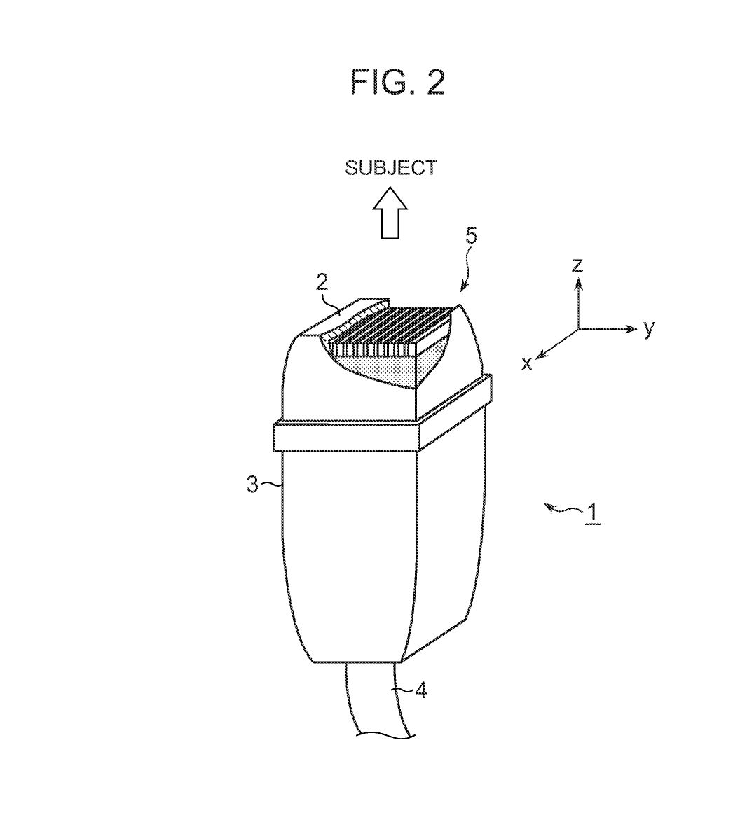

The ultrasonic probe 1 will be described based on FIGS. 2, 3 and 4. The ultrasonic probe 1 ultrasonically scans a subject and receives an ultrasonic echo signal.

The ultrasonic probe 1 has an acoustic lens section 2 at an end portion thereof. The ultrasonic probe 1 is provided with a probe housing 3 and a connection cable 4 for connection to the apparatus main body 101.

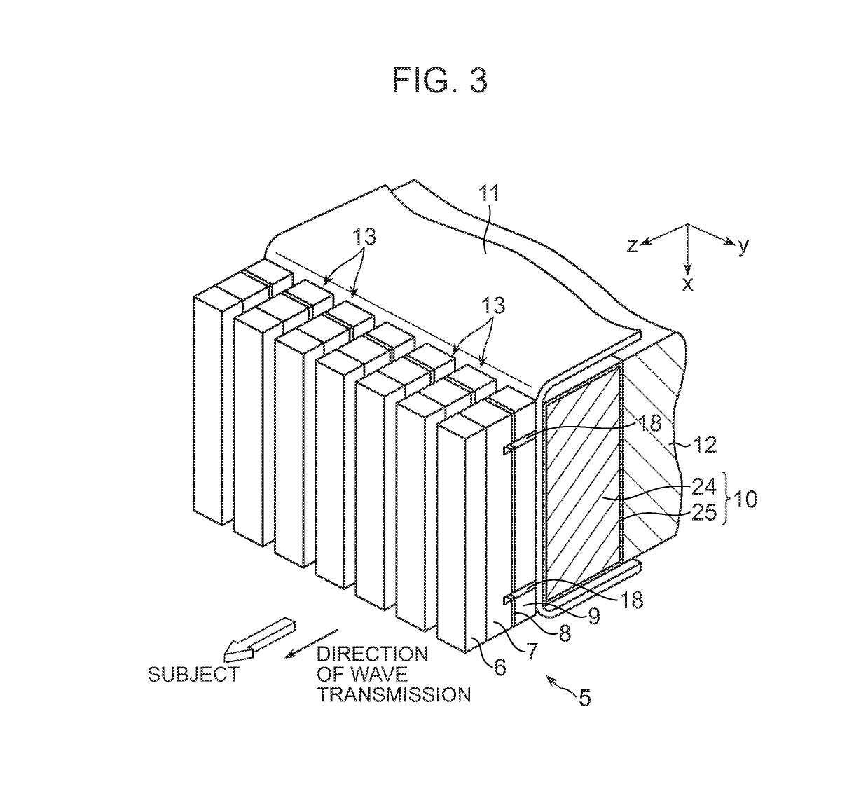

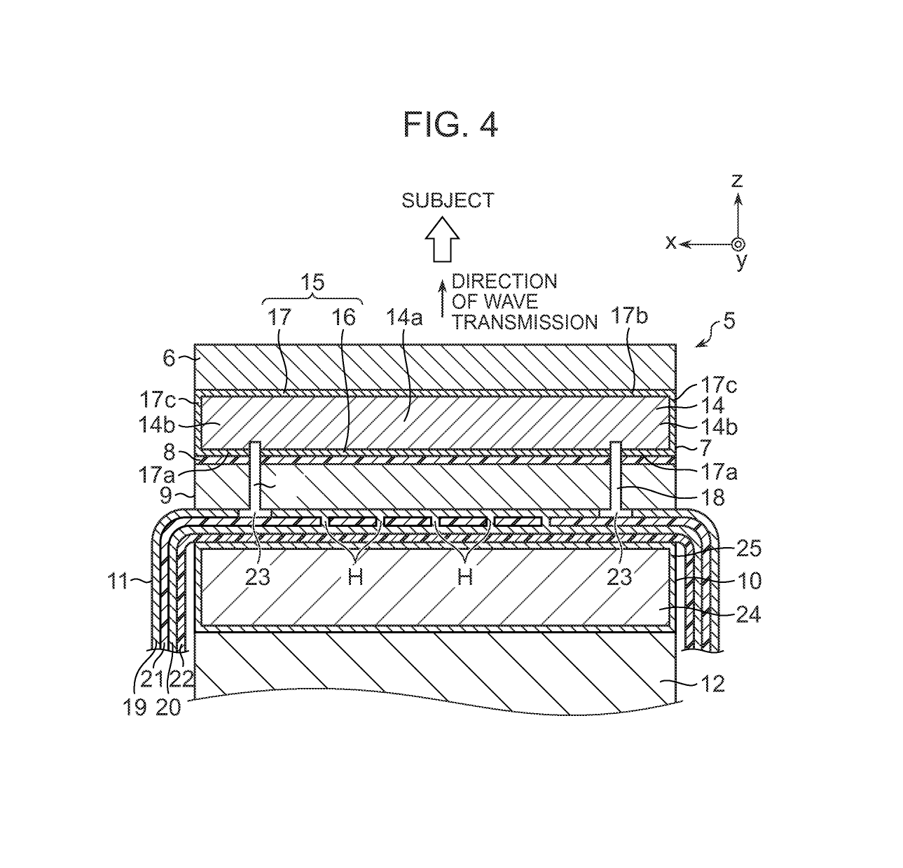

The probe housing 3 is internally provided with a functional element section 5. The functional element section 5 will be described in the following in detail based on FIGS. 3 and 4. The functional element section 5 is provided with acoustic matching layers 6, ultrasonic transducers 7, adhesive layers 8, reflection layers 9, a backing layer 10, a flexible substrate 11 and a metallic body 12. The acoustic matching layers 6, ultrasonic transducers 7 and reflection layers 9 each have a parallelepiped shape extending in the x-axis direction with one of each of them stacked in the z-axis direction, along which ultrasonic waves are transmitted, to form a laminated structure 13 such that plural laminated structures 13 are arrayed in the y-axis direction.

Each of the acoustic matching layers 6 is bonded to the surface on the side through which ultrasonic waves are transmitted of the adjoining ultrasonic transducer 7 (adhesive layer not shown). The acoustic matching layer 6 has an impedance between the impedances of the ultrasonic transducer 7 and the acoustic lens section 2. The acoustic matching layer 6 has a thickness approximately equaling one quarter of the center-frequency wavelength of the ultrasonic waves transmitted therethrough and inhibits the ultrasonic waves from being reflected at an interface with a different acoustic impedance. Even though, in the present example, the acoustic matching layer is shown as a singular layer, the acoustic matching layer may alternatively include a plural layer structure.

The ultrasonic transducer 7 has a piezoelectric material 14 and a conductive layer 15. The piezoelectric material 14 is, for example, piezoelectric zirconate titanate (PZT). The conductive layer 15 is formed over the surface of the piezoelectric material 14, for example, by sputtering.

The conductive layer 15 has a signal electrode 16 and a ground electrode 17. The signal electrode 16 is formed in a portion 14a, between bores 18 being described later, of the piezoelectric material 14. The ground electrode 17 includes first portions 17a, a second portion 17b and third portions 17c. The first portions 17a are formed in end portions 14b of the piezoelectric material 14 to be in the same plane as the signal electrode 16 separated from the first portions 17a by the bores 18. The second portion 17b is formed over the surface, opposite to the surface where the first portions 17a are formed, of the piezoelectric material 14. The third portions 17c are formed over side surfaces between the first portions 17a and the second portion 17b of the parallelepiped ultrasonic transducer 7. The signal electrode 16 is formed between the first portions 17a of the ground electrode 17. The signal electrode 16 and the ground electrode 17 are electrically isolated from each other by the bores 18.

The total thickness of the ultrasonic transducer 7 and the adhesive layer 8 approximately equals one quarter of the center-frequency wavelength of the ultrasonic waves generated by the vibration of the ultrasonic transducer 7. To be specific, the ultrasonic transducer 7 has a thickness of about several hundred microns.

The reflection layer 9 is bonded to the surface, opposite to the subject side (i.e. opposite to the acoustic matching layer 6), of the ultrasonic transducer 7 by the adhesive layer 8 of, for example, an epoxy resin adhesive. Namely, the reflection layer 9 is bonded to the signal electrode 16 and the first portions 17a.

The surface on the ultrasonic transducer 7 side of the reflection layer 9 is mirror-polished. The surfaces of the signal electrode 16 and the first portions 17a formed over the ultrasonic transducer 7 are also mirror-polished. The surface roughness of the mirror-polished surface, on the ultrasonic transducer 7 side, of the reflection layer 9 and the mirror-polished surfaces of the signal electrode 16 and the first portions 17a formed over the ultrasonic transducer 7 is held to be about several microns. Thus, it is possible to make the adhesive layer 8 as uniformly thin as possible, for example, to be about several microns thick.

As described above, the thickness of the adhesive layer 8 is about the same as the surface roughness of each of the signal electrode 16, the first portions 17a and the reflection layer 9. In such a condition, even though the adhesive layer 8 is an insulator containing an epoxy resin adhesive, the signal electrode 16 and the first portions 17a are partially, at irregular surface portions, in contact, to be electrically connected, with the reflection layer 9.

The reflection layer 9 functions as a fixed plate which reflects, toward the subject, the ultrasonic waves coming from the ultrasonic transducer 7 after being generated by the vibration of the ultrasonic transducer 7. The ultrasonic waves reflected by the reflection layer 9 increase the ultrasonic power incident on the subject. The reflection layer 9 represents an exemplary embodiment of the reflection layer. The reflection layer 9 designed to reflect the ultrasonic waves coming from the ultrasonic transducer 7 is made of a material with an acoustic impedance higher than that of the piezoelectric body 14. The reflection layer 9 is made of, for example, tungsten.

Since tungsten, of which the reflection layer 9 is made, is conductive, the reflection layer 9 has a function to electrically connect a first copper foil layer 19 and a second copper foil layer 20, being described later, of the flexible substrate 11 with the signal electrode 16 and the ground electrode 17 of the ultrasonic transducer 7. This allows the voltage supplied from the first copper foil layer 19 and the second copper foil layer 20 to be applied to the ultrasonic transducer 7 via the reflection layer 9.

The bores 18 are provided in longitudinal end portions on both sides of the reflection layer 9, the adhesive layer 8 and the ultrasonic transducer 7. The bores 18 are formed by performing cutting work using, for example, a diamond whetstone applied, from the reflection layer 9 side, to the ultrasonic transducer 7 and the reflection layer 9 having been bonded together by the adhesive layer 8.

To the surface, opposite to the surface bonded to the ultrasonic transducer 7, of the reflection layer 9, the flexible substrate 11 is bonded using an adhesive such that the flexible substrate 11 is between the reflection layer 9 and the backing layer 10 (adhesive layer not shown). The flexible substrate 11 externally extends in the thickness direction, along side surfaces, of the backing layer 10 to be connected to the connection cable 4 (connection structure not shown).

The structure of the flexible substrate 11 will be described in the following. The flexible substrate 11 has the first copper foil layer 19, the second copper foil layer 20, a first polyimide film layer 21 and a second polyimide film layer 22. The first copper foil layer 19 and the second copper foil layer 20 are insulated from each other by the first polyimide film layer 21. The first copper foil layer 19 is formed to be closer, in the state bonded to the reflection layer 9, to both ends of the reflection layer 9 than the bores 18 are. The second copper foil layer 20 is laminatedly sandwiched between the first polyimide film layer 21 and the second polyimide film layer 22 while having an outer portion formed to be present, via through-holes H, in a center surface portion, between the bores 18, of the reflection layer 9 and in the same plane as the first copper foil layer 19. The first copper foil layer 19 and the portion of the second copper foil layer 20 present in the same plane are insulated from each other by separation grooves 23. The separation grooves 23 are formed to be aligned with the bores 18 in a state with the flexible substrate 11 bonded to the reflection layer 9. In this arrangement, the first copper foil layer 19 is electrically connected to the end portions, closer to both ends than the bores 18 are, of the conductive reflection layer 9, whereas the second copper foil layer 20 is electrically connected to the center portion, between the bores 18, of the reflection layer 9. Thus, the first copper foil layer 19 is electrically connected, via the reflection layer 9, with the first portions 17a of the ground electrode 17 included in the ultrasonic transducer 7, and the second copper foil layer 20 is electrically connected, via the reflection layer 9, with the signal electrode 16 included in the ultrasonic transducer 7.

The first copper foil layer 19 connected with the ground electrode 17 is formed to extend along the whole length of the flexible substrate 11, so that it is connected commonly with all the ultrasonic transducers 7 arrayed in the y-axis direction. The second copper foil layer 20, on the other hand, is divided by copper foil dividing grooves into plural parts along the y-axis direction forming plural copper foil patterns, not shown, in the flexible substrate 11. The plural copper foil patterns correspond to the plural laminated structures arrayed along the y-axis direction.

The backing layer 10 is bonded to the flexible substrate 11 or formed directly on the back side of flexible substrate 11 to hold the flexible substrate 11. The backing layer 10 represents an exemplary embodiment of the backing layer.

The backing layer 10 has a backing material 24 and a thermally-conductive layer 25. The backing material 24 is, for example, an epoxy resin formed by dispersing and solidifying metallic powder. The thermally-conductive layer 25 is formed over the surface of the backing material 24. The thermally conductive layer 25 is made of a material having a thermal conductivity higher than that of the backing material. For example, it is formed by coating the surface of the backing material 24 with sheet metal. Forming the thermally-conductive layer 25 by coating the backing material 24 with sheet metal makes formation of the thermally-conductive layer 25 easy.

As long as the thermally-conductive layer 25 has a thermal conductivity several hundred to several thousand times higher than that of the backing material 24, it need not be metallic. The thermally-conductive layer 25 may be formed of, for example, carbon.

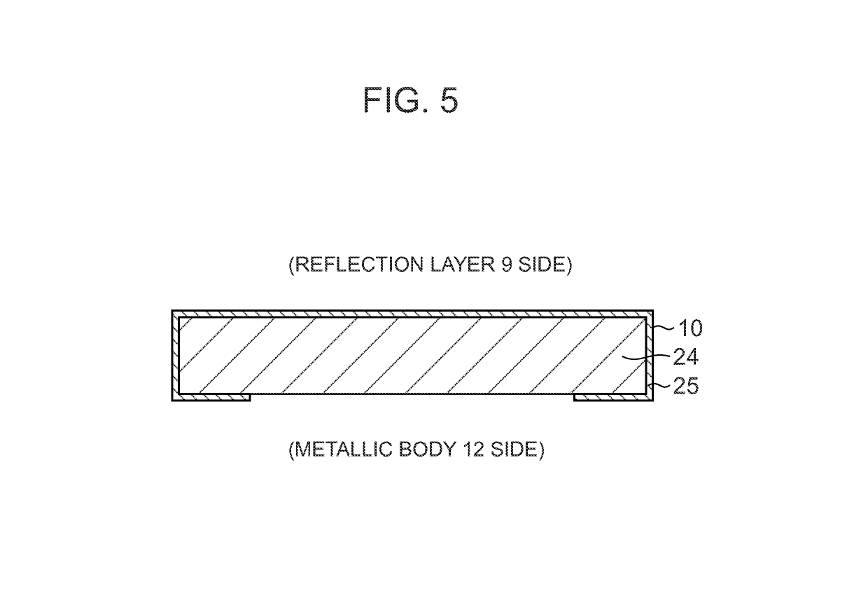

Even though, in the present example, the thermally-conductive layer 25 is formed over the whole surface of the backing layer 10, the thermally-conductive layer is at least required to be formed to cover the surface, on the reflection layer 9 side, of the backing layer 10 and extend to reach the opposite surface, on the metallic body 12 side, of the backing layer 10. For example, as shown in FIG. 5, the thermally-conductive layer 25 need not cover the whole surface on the metallic body 12 side of the backing layer 10 as long as it is formed to cover both end portions along the x-axis direction of the surface on the metallic body 12 side of the backing layer 10.

In the exemplary embodiment, the thermally-conductive layer 25 has a thickness not exceeding 10% of the center-frequency wavelength of the ultrasonic waves transmitted from the ultrasonic transducer 7. This is for the following reason. Most of the ultrasonic waves transmitted from the ultrasonic transducer 7 to the reflection layer 9 side (opposite to the subject side) are reflected by the reflection layer 9 toward the subject side. Low-frequency ultrasonic waves, however, pass the reflection layer 9 and reach the backing material 24 to be absorbed thereby.

If the thermally-conductive layer 25 is too thick, the ultrasonic waves transmitted through the reflection layer 9 may possibly be reflected by the thermally-conductive layer 25 before being absorbed by the backing material 24. When the thickness of the thermally-conductive layer 25 is as described above, ultrasonic waves can be inhibited from being reflected by the thermally-conductive layer 25.

The metallic body 12 is bonded to the backing layer 10 using an adhesive (the adhesive layer is not shown). The metallic body 12 makes up, for example, a portion of the probe housing 3.

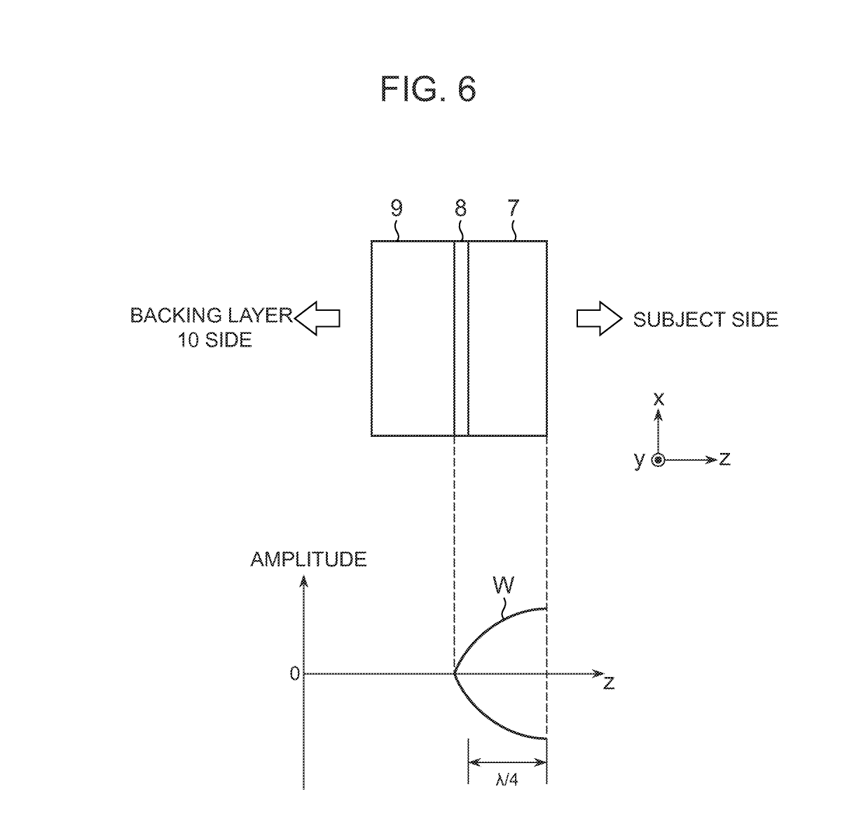

The operation of the functional element section 5 included in the ultrasonic probe 1 of the present example will be described in the following. The ultrasonic transducer 7 excites resonant vibration when a voltage is applied between the signal electrode 16 and the ground electrode 17. With the acoustic matching layer 6 of a low acoustic impedance present on the subject side and the reflection layer 9 of a high acoustic impedance present on the backing layer 10 side opposite to the subject side, the resonant vibration forms, as shown in FIG. 6, a standing wave W having a free end on the subject side and a fixed end on the reflection layer 9 side.

Note that the coordinate positions along the z axis shown in FIG. 6 correspond to the positions along the z axis of the ultrasonic transducer 7 and the reflection layer 9 shown in FIG. 4.

FIG. 6 shows a standing wave W the amplitude of which is maximum at the surface on the subject side of the ultrasonic transducer 7 and is zero at the surface on the ultrasonic transducer 7 side of the reflection layer 9. The reflection layer 9 functions as a fixed end. Thus, in the ultrasonic transducer 7 in a resonant condition, a standing wave W one quarter of whose wavelength equals the thickness in the z-axis direction of the ultrasonic transducer 7 is generated.

Since the adhesive layer 8 is uniformly thin as described above, the adhesive layer 8 does not prevent the reflection layer 9 from functioning as a fixed end.

The heat generated in the ultrasonic transducer 7 when transmitting ultrasonic waves reaches the backing layer 10 via the reflection layer 9 and the flexible substrate 11. The heat having reached the backing layer 10 reaches the metallic body 12 via the thermally-conductive layer 25.

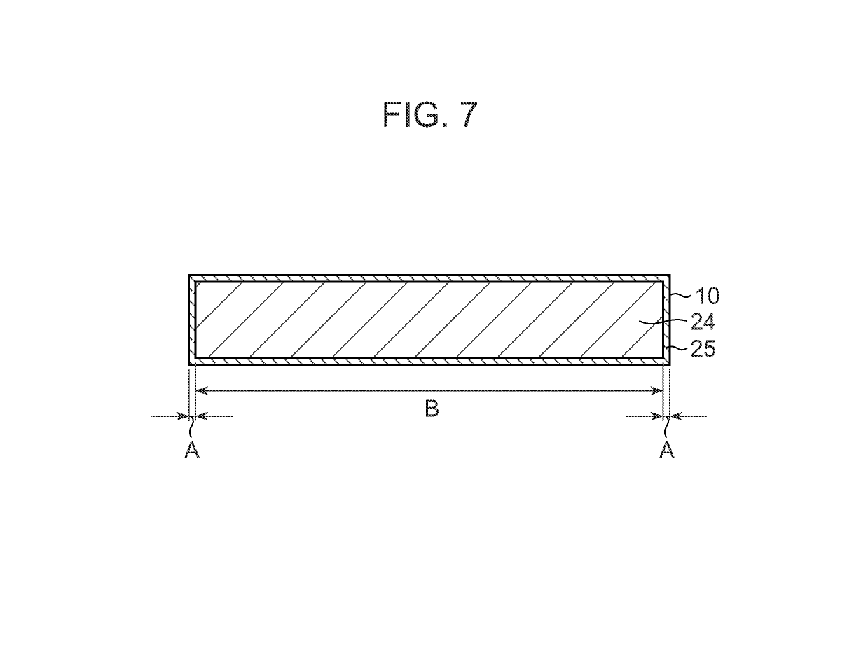

The thermally-conductive layer 25 has a thermal conductivity several hundred to several thousand times higher than that of the backing material 24. As shown in FIG. 7 in which the width (thickness) of the thermally-conductive layer 25 is denoted as “A” and the width in the x-axis direction of the backing material 24 is denoted as “B,” the width (2×A) in the x-axis direction of the thermally-conductive layer 25 is several hundred times smaller than the width B in the x-axis direction of the backing material 24. Even though the width of the thermally-conductive layer 25 is small compared with the backing material 24, with the thermally-conductive layer 25 having a thermal conductivity several hundred to several thousand times higher than that of the backing material 24, the thermal conductivity of the backing material 24 provided with the thermally-conductive layer 25 is several times to several hundred times higher than that of the backing material 24 without the thermally-conductive layer 25. Thus, the heat generated in the ultrasonic transducer 7 is conducted to the metallic body 12 on the side opposite to the subject side more easily than in prior-art cases where the backing material 24 is not provided with the thermally-conductive layer 25. This can prevent the acoustic output from being constrained by the surface temperature of the acoustic lens section 2.

Since the ultrasonic waves transmitted from the ultrasonic transducer 7 oppositely to the subject side are reflected by the reflection layer 9, the thermally-conductive layer 25, that may be metallic, formed over the surface of the backing layer 10 does not generate any acoustically undesired effect.

Exemplary embodiments have been described above, but it is needless to say that various changes and modifications can be made in the invention within the scope not departing from the spirit thereof For example, even though the thermally-conductive layer 25 is formed by coating the backing material 24 with sheet metal, it may be formed by a different method. For example, the thermally-conductive layer 25 may be formed by applying a plating process to the surface of the backing material 24.

1. An ultrasonic probe comprising a reflection layer between an ultrasonic transducer and a backing layer, the reflection layer configured to reflect an ultrasonic wave transmitted from the ultrasonic transducer, wherein the backing layer includes a backing material, and wherein a thermally-conductive layer of material having a thermal conductivity higher than that of the backing material is formed over a surface of the backing layer.

2. The ultrasonic probe according to claim 1, wherein the thermally-conductive layer extends from the surface on a reflection layer side of the backing material to a surface opposite to the reflection layer side of the backing material.

3. The ultrasonic probe according to claim 1, wherein the thermally-conductive layer has a thickness not exceeding 10% of a center frequency wavelength of the ultrasonic wave transmitted from the ultrasonic transducer.

4. The ultrasonic probe according to claim 2, wherein the thermally-conductive layer has a thickness not exceeding 10% of a center frequency wavelength of the ultrasonic wave transmitted from the ultrasonic transducer.

5. The ultrasonic probe according to claim 1, wherein the thermally-conductive layer is formed by coating the surface of the backing material with a sheet of material having a thermal conductivity higher than that of the backing material.

6. The ultrasonic probe according to claim 1, further comprising a metallic body which is in contact with a surface opposite to a reflection layer side of the backing layer.

7. The ultrasonic probe according to claim 1, wherein the reflection layer has an acoustic impedance greater than that of the ultrasonic transducer and functions as a fixed end to reflect the ultrasonic wave transmitted from the ultrasonic transducer.

8. The ultrasonic probe according to claim 1, wherein the thermally-conductive layer is made of one of metal and carbon.

9. An ultrasonic display device comprising the ultrasonic probe according to claim 1.

10. An ultrasonic display device comprising the ultrasonic probe according to claim 2.

11. An ultrasonic display device comprising the ultrasonic probe according to claim 3.

12. An ultrasonic display device comprising the ultrasonic probe according to claim 4.

13. An ultrasonic display device comprising the ultrasonic probe according to claim 5.

14. An ultrasonic display device comprising the ultrasonic probe according to claim 6.

15. An ultrasonic display device comprising the ultrasonic probe according to claim 7.

16. An ultrasonic display device comprising the ultrasonic probe according to claim 8.

17. A method of assembling an ultrasonic probe, said method comprising:
providing an ultrasonic transducer;
providing a backing layer including a backing material; and
positioning a reflection layer between the ultrasonic transducer and the backing layer, wherein the reflection layer is configured to reflect an ultrasonic wave transmitted from the ultrasonic transducer; and
forming a thermally-conductive layer of material having a thermal conductivity higher than that of the backing material over a surface of the backing layer.

18. A method in accordance with claim 17, wherein forming a thermally conductive layer comprises coating the surface of the backing material with a sheet of material having a thermal conductivity higher than that of the backing material.

19. A method in accordance with claim 17, wherein forming a thermally conductive layer comprises forming the thermally conductive layer such that the thermally-conductive layer extends from the surface on a reflection layer side of the backing material to a surface opposite to the reflection layer side of the backing material.

20. A method in accordance with claim 17, wherein forming a thermally conductive layer comprises forming the thermally conductive layer such that the thermally-conductive layer has a thickness not exceeding 10% of a center frequency wavelength of the ultrasonic wave transmitted from the ultrasonic transducer.
